(19) United States
(12) Patent Application Publication
(10) Pub. No.: US 2002/0037521 A1
(43) Pub. Date: Mar. 28, 2002

(54) Method of identifying the source of genetic information in DNA

(75) Inventors: Hisashi Kashima, Yamato-shi (JP); Harunobu Kudo, Yamato-shi (JP); Ryoh Sugihara, Yamato-shi (JP)

Correspondence Address: Casey August, IBM Corporation, Intellectual Propety Law Dept., P.O. Box 218, Yorktown Heights, NY, 10598, US

(73) Assignee: International Business Machines Corporation, Armonk, NY

(21) Appl. No.: 09/870,009

(22) Filed: May 30, 2001

(30) Foreign Application Priority Data
Jun. 7, 2000 (JP) 2000-171030

(51) Int. Cl. C12Q 1/68; C12P 19/34; H04L 9/00

(52) U.S. Cl. 435/6; 435/91.2; 380/42

(57) ABSTRACT

The present invention embeds predetermined information in the nucleotide sequence of DNA to identify the source of the genetic information in DNA. In particular, the pattern of a nucleotide sequence that normally does not appear in DNA is correlated with identification information for identifying a source of predetermined genetic information belonging to the DNA, and, in said portion of the DNA other than the gene, the nucleotide sequence that is correlated with the identification information is embedded in the DNA so as not to affect the genetic information in the DNA.

### FIELD OF THE INVENTION

The present invention relates to a method for embedding watermark information in DNA to identify the source of genetic information provided for the DNA.

### BACKGROUND OF THE INVENTION

Of the various plants and animals found on earth, there are organisms such as soy beans, which have acquired a natural resistance to noxious insects, that possess qualities that may be considered superior when compared with those of others of the same species. Further, there are organisms such as racehorses, valued as the offspring of good breeding stock, for which worth is assigned based on an artificial evaluation reference. When these properties and values are rated and levels are assigned to the genes that produce them, the genes are credited with providing an added value, as being “value-added genes”. And even today, such so-called value-added genes are being traded for money. For example, an organism credited with having one of these value-added genes normally fetches a higher price than does another that is not so credited.

While value-added genes may be produced as a result of natural selection, in most cases today, artificial, intentional manipulation is employed to develop or generate such genes. And it is anticipated that as the development of life sciences continues, the intentional production, through artificial manipulation, of value-added genes (and of organisms in which value-added genes are dominant) can only increase.

In this case, for economical reasons a producer who has developed a value-added gene may not wish that it be freely available for third party use. For example, a producer holding the original genetic information for a value-added gene may permit a third party to use the gene under conditions whereby its employment is limited to a single generation, i.e., a condition whereby copying, including breeding or cultivating or copying at the DNA (deoxyribonucleic acid) or the RNA (ribonucleic acid) level, is inhibited.

However, the copying of plants or animals using genetic information can be performed by gathering spermatozoa or seeds, without highly technical or expensive apparatuses being required. Further, when bioengineering techniques are employed, the high-level copying of genetic information can be performed at the DNA or the RNA level.

As is described above, the genetic information carried by plants and animals can be copied without using highly technical or expensive apparatuses, and by using the techniques embodied in bioengineering, the high-level copying of genetic information at the DNA or RNA level can also be performed. Therefore, it is very difficult to apply technical restrictions to the copying, by third parties, of the above described value-added genes.

Further, when a value-added gene generated by a predetermined producer is detected in a specific organism, it is difficult to determine whether the gene was illegally copied, because it is hard to distinguish copying from gene mutation.

If predetermined information, such as ID information, can be embedded in the DNA nucleotide sequence of a specific value-added gene, when the value-added gene is copied the ID information will also be copied. Therefore, when an examination is made to decide whether the DNA nucleotide sequence of an organism having a value-added gene includes ID information, a determination can be made as to whether the value-added gene was obtained by copying.

It is, therefore, one object of the present invention to embed predetermined information in the nucleotide sequence of DNA and to identify the source of the genetic information in DNA.

It is another object of the present invention to detect and analyze information that is intentionally embedded in the sequence of nucleotides making up DNA and to determine whether a predetermined gene owned by a predetermined organism is a copy of a specific gene.

It is an additional object of the present invention to provide means that can determine whether a predetermined gene owned by a predetermined organism is a copy of a specific gene, and to thus prevent the illegal copying of the specific gene by a third party.

### SUMMARY OF THE INVENTION

To achieve these objects, according to the present invention, the following method for writing information in DNA is provided. Specifically, a method for writing information in DNA comprises the steps of: correlating the pattern of a nucleotide sequence, which normally does not appear in a portion of the DNA other than a gene, with identification information for identifying a source of predetermined genetic information belonging to the DNA; and embedding, in the portion of the DNA other than the gene, the nucleotide sequence that is correlated with the identification information. When the pattern of the nucleotide sequence does not normally appear in a portion other than a gene, it means that it is stochastically ensured that under normal conditions this pattern is not present in a portion of DNA other than a gene. This probability can be calculated by a statistic process using frequency distribution.

According to the present invention, a method is provided for writing information in the gene portion of a DNA molecule, instead of in the other portion. Specifically, the method for writing information in DNA comprises the steps of: correlating the pattern of a nucleotide sequence, which normally does not appear in the intron of a DNA, with identification information for identifying the source of predetermined genetic information owned by the DNA; and embedding, in the intron of the DNA, the nucleotide sequence that is correlated with the identification information. When the pattern of the nucleotide sequence does not normally appear in the intron, it means that it is stochastically ensured that under normal conditions this pattern is not present in the intron of DNA. This probability can be calculated by employing a statistic process for which frequency distribution is used.

Further, according to the present invention, a method for writing information in an exon of DNA, including genetic information. That is, the method for writing information in DNA comprises the steps of: employing redundancy for a codon to be translated into amino acid so that multiple codons to be translated into the same amino acid are correlated with binary data; and arranging, in the exon of a gene, the codons that are correlated with the binary data, and to thus form a data sequence representing predetermined information. With this configuration, the genetic information and the binary data are multiplexed and included in an array of codons.

As for codon redundancy, even when the action of codons, relative to the kinds of amino acid into which the codons are to be translated, is the same, the use of codons varies, depending on the species of an organism, and is normally biased. Therefore, in order to restrict the influence on the organism as much as possible, it is preferable that frequently employed codons be selected for a targeted organism for information embedding, and that they be correlated with binary data.

Further, according to the present invention, a method is provided for employing the information thus inserted into DNA to identify the source of genetic information in DNA that has been obtained from a predetermined organism. Specifically, this method comprises the steps of: obtaining DNA from an arbitrary organism of the same species as an organism wherein a source identification nucleotide sequence, for designating the source of genetic information, is embedded into the DNA; and employing as the source identification nucleotide sequence a complementary nucleotide sequence in order to determine whether the source identification nucleotide sequence is present in the DNA of the arbitrary organism.

Furthermore, according to the present invention, a DNA is provided to which information is added by the above information writing method. Specifically, this DNA comprises: a gene portion including genetic information; and a portion, other than the gene portion, including no genetic information, wherein the portion other than the gene portion includes a nucleotide sequence that is correlated with source identification information and specifies a source of genetic information that is transmitted by the gene portion.

The gene portion that includes genetic information also includes exon that is translated into amino acid when protein is to be synthesized, and intron that is removed when protein is to be synthesized, and the intron includes a nucleotide sequence that is correlated with source identification information for designating a source of genetic information that is included in the exon.

DNA includes multiple kinds of codons that are correlated with the binary data using the codon redundancy and are translated into amino acid, and binary data are used to correlate the codon array in the gene portion with a data sequence that represents predetermined information.

DNA is provided wherein a special sequence that is intentionally designed is included as a part of a nucleotide sequence, wherein the special sequence is correlated with source identification information for designating the source of genetic information included in the DNA, and wherein the special sequence is embedded in the DNA so as not to affect the transmission of the genetic information included in the DNA.

For the DNA to which these data are added, multiple special sequences (nucleotide sequences correlated with information) are repetitively embedded, or multiple kinds of special sequences are embedded, in portions other than the gene portions or in corresponding locations, such as introns and exons.

Since multiple special sequences, or multiple kinds of special sequences are embedded, the probability that a special sequence will be naturally destroyed or will be naturally generated though the mating process can be reduced.

In addition, the present invention can be provided as a nucleotide sequence that is designed to add information to DNA, or the cell of an organism that includes DNA to which information has been added.

### BRIEF DESCRIPTION OF THE DRAWINGS

FIG. 1 is a diagram for explaining the process for synthesizing genes in a DNA to obtain protein.

FIG. 2 is a flowchart for explaining the general processing according to this invention used to determine a watermark sequence, by embedding it in a DNA and detecting it therein.

FIG. 3 is a diagram for explaining the concept of a method for inserting a watermark sequence according to a first embodiment of the present invention.

FIG. 4 is a flowchart showing the processing according to the first embodiment used for calculating an appearance probability of a sequence based on DNA sequence data, and for determining a proposed watermark sequence.

FIG. 5 is a diagram showing an example frequency distribution, using pseudo data, for a nucleotide sequence having six bases in one organism.

FIG. 6 is a diagram showing an example frequency distribution graph of the number of organisms relative to the appearance frequency of a nucleotide sequence AAAGTC in FIG. 5.

FIG. 7 is a flowchart showing the processing for confirming the safety of a watermark sequence according to the first embodiment.

FIG. 8 is a diagram showing the state wherein a watermark sequence is detected by using a complementary nucleotide sequence.

FIG. 9 is a diagram showing the state wherein the nucleotide sequence of a DNA is read using a sequencer, and a watermark sequence is detected.

FIG. 10 is a diagram for explaining the concept of a method for inserting a watermark sequence in accordance with a second embodiment of the present invention.

FIG. 11 is a diagram for explaining the concept of a method for inserting a watermark sequence in accordance with a third embodiment of the present invention.

FIG. 12 is a table showing the toleration for the first to the third embodiments relative to the individual copying methods.

FIG. 13 is a table showing codons and corresponding amino acids (or special meanings).

### DESCRIPTION OF THE PREFERRED EMBODIMENTS

The preferred embodiments of the present invention will now be described in detail while referring to the accompanying drawings.

First, an overview of the present invention will be given. According to the present invention, a nucleotide sequence carrying predetermined information, such as ID information, is embedded in DNA, so that DNA including such a nucleotide sequence is distinguishable. Thereafter, the nucleotide sequence carrying this predetermined information is called a watermark sequence, and the information represented by the watermark sequence is called watermark information. When this watermark sequence is embedded in DNA including a value-added gene that is provided by selective breeding or through gene manipulation, if the value-added gene is copied during the breeding process by employing the various other methods, the source of the genetic information in the gene can be identified. And if the watermark sequence is detected in the DNA of a predetermined organism, it can be ascertained that the gene of the organism is a copy of the DNA wherein the watermark was previously embedded, and is not one that is naturally generated through gene mutation. With this watermarking method, even when a value-added gene is copied, it can be determined whether the copying was performed legally or illegally.

The specific procedures performed when embedding the watermark sequence are as follows.

(1) A watermark sequence W is embedded in the DNA of a germ cell, such as a spermatozoon, an ovum or a zygote carrying superior genetic information I (where W represents a spermatozoon and A represents an ovum).

(2) A is fertilized, grows and becomes an image A′.

(3) Imago A′ copies it own genetic information to form B (a spermatozoon or an ovum).

(4) B is fertilized, grows and becomes an imago B′.

(5) When the watermark sequence W is detected in the DNA of imago B′, it can be determined that imago B′ includes a copy of the genetic information I.

As the copying of the gene is repeated during mating or as an extended time elapses, the genetic information I is degraded. Therefore, when the genetic information I is so degraded that the watermark sequence W can not be detected in the DNA, it can be ascertained that the value of the genetic information I has also been degraded.

According to the invention, recognition of watermarked DNA is possible only when a watermark sequence is present. That is, it is not anticipated that watermark information in a watermark sequence will have a specific effect on and alter an organism that includes the DNA in question. Therefore, the present invention can be employed for all species, including plants and animals.

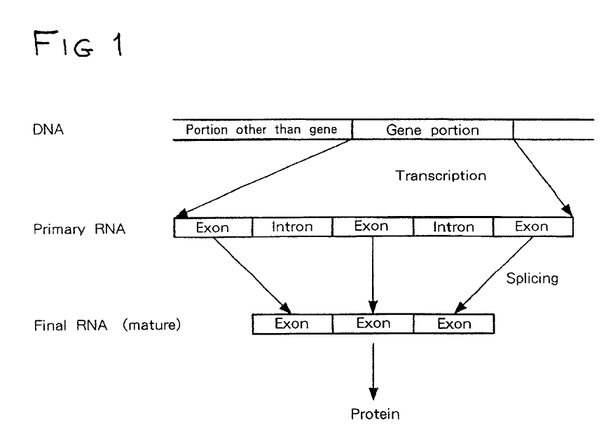

The form of the genetic information in a cell will now be described through an explanation of the overview of a process by which gene codes for a protein molecule. FIG. 1 is a diagram for explaining a process for synthesizing a gene in DNA to obtain protein. Arranged in the DNA are four bases, A (adenine), T (thymine), G (guanine) and C (cytosine). This sequence of the four bases (hereinafter the bases are referred to by their initials, A, T, G and C) of DNA consists of a gene portion wherein a protein code sequence and its transcription control information are stored, and a portion wherein genetic information is not included. As is shown in FIG. 1, through the transcription process employed for the synthesization of protein, only the gene portion pertinent to the protein is transcribed as an intermediate genetic material called mRNA. In the case of a higher organism, the mRNA consists of exon, which is finally translated into amino acid, and intron, which is removed during the process (this state is called the primary mRNA). Finally, the intron is removed (splicing), and the final mRNA (mature mRNA) is obtained. The final mRNA is then translated and coded for protein.

Now, a method for copying the genetic information will be explained. Technically different methods for copying genetic information can be employed in accordance with the physical storage of genetic information. For example, if the genetic information is coded unchanged with the DNA form, an inexpensive and easy method, such as breeding or cultivating, or a method for extracting one region from the DNA, including the gene, can be employed. Further, a method for copying the genetic information from another state, such as RNA, or a method for reading a sequence of genes and synthesizing them, can also be employed.

To determine the source of genetic information using the watermark information, the watermark information should be able to tolerate the copying process performed by the various methods mentioned above. When the watermark information can tolerate the copying, it means that the watermark sequence can be maintained even after the gene is copied, and can thus be detected. As is described above, since various technically different methods can be employed when copying genetic information, these copying methods should be taken into consideration for the embedding of a watermark sequence in DNA, in order to provide copy toleration for the watermark information. In the present invention, the following three methods are proposed while also taking into account the safety of the watermark sequence, which will be described later:

a) a method for inserting a watermark sequence to the portion of the DNA other than the gene portion;

b) a method for inserting a watermark sequence into the intron of the gene to be protected; and

c) a method for embedding watermark information by using the codon redundancy.

A further description will be given here of the method by which codon redundancy is used to embed watermark information.

As is described above, the DNA consists of the four bases A, C, G and T. When the DNA is transcribed into RNA, thymine (T) is replaced by uracil (hereinafter referred to as U), and thus, RNA consists of a sequence of the four bases A, C, G and U. For the transformation of the bases into amino acid, codon consisting of a set of three of the bases A, C, G and U is employed as a unit.

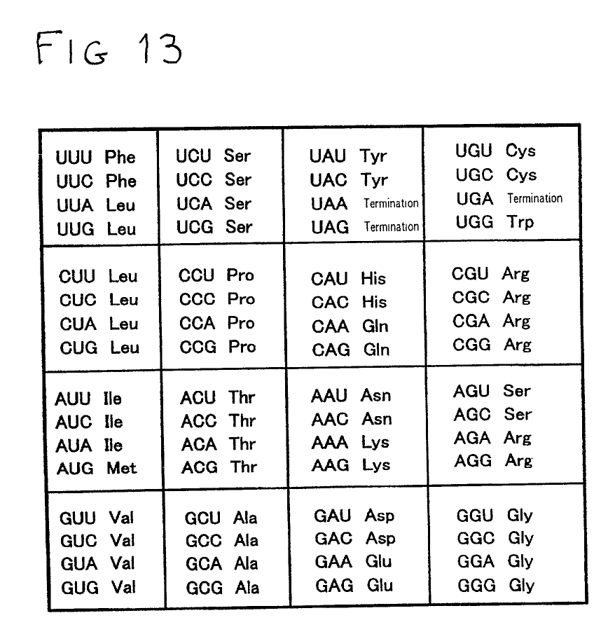

FIG. 13 is a table showing codons and corresponding amino acids (or special definitions). In the table in FIG. 13 (hereinafter referred to as a codon table), codons are arranged in the left columns, and amino acids (or special definitions) are arranged in the right columns and are represented by their abbreviations: phenylalanine (Phe), leucine (Leu), serine (Ser), tyrosine (Tyr), cystine (Cys), tryptophan (Trp), proline (Pro), histiline (His), glutamine (Gln), Arginine (Arg), isoleucine (Ile), methionine (Met), threonine (Thr), asparagine (Asn) lysine (Lys), valine (Val), alanine (Ala), aspartic acid (Asp), glutamic acid (Glu) and glycine (Gly). Note that “termination” indicates that the process for the translation of a codon into amino acid is terminated.

As is apparent from the codon table, codon does not have a one-to-one correspondence with amino acid, and there are multiple codons that can be translated into one amino acid. This redundancy means that even when a sequence differs at the RNA level (or in the DNA before transcription), at the final amino acid level the same material is obtained by synthesization.

Since for an organism all that is necessary is that amino acid be correctly generated by synthesization, at the DNA or RNA level an arbitrary codon within this redundancy range can be selected. When this fact is employed and a codon representing a predetermined gene is intentionally selected, watermark information can be written.

An explanation will now be given for a condition that permits a nucleotide sequence to be employed as a watermark sequence. In order to embed a predetermined nucleotide sequence in DNA as a watermark sequence, the nucleotide sequence must be safe and must serve as a probative force. When the nucleotide sequence is safe, it means that the nucleotide sequence is not significant as a source form, i.e., an organism is not affected by the insertion in DNA of a watermark sequence. The procedures for confirming the safety of a food, for example, must be performed in accordance with general standards, such as the “Guideline for evaluation of the safety of recombinant DNA techniques for foods and food additives” established by the Ministry of Health and Welfare. Furthermore, while taking safety into account, the position whereat the watermark sequence can be inserted is limited to a portion of the DNA that is not biologically significant. Therefore, as is described above, a portion of DNA other than a gene, or the intron of a gene, is selected as the portion wherein the watermark sequence is embedded in DNA. It should be noted that when the codon redundancy is employed, safety is ensured, so that watermark information can be embedded in an exon that is biologically significant.

When the nucleotide sequence includes probative force, it means that it is guaranteed that detection of the watermark sequence indicates that copying was performed. That is, a sequence that corresponds to the watermark sequence should not originally be present in DNA, or should not occur naturally due to a slight change in the DNA. To implement this probative force, it must be stochastically guaranteed that the same sequence as the watermark sequence does not appear naturally. Therefore, the size of the watermark sequence, the number of watermark sequences, the type of the watermark sequence and the insertion location must be taken into consideration. The setting of these parameters will be specifically described later. When the codon redundancy is employed to embed watermark information, a rare combination of codons should be employed to write the watermark information. This can prove that the sequence was not coincidentally inserted into a gene, but was intentionally inserted in the gene as watermark information.

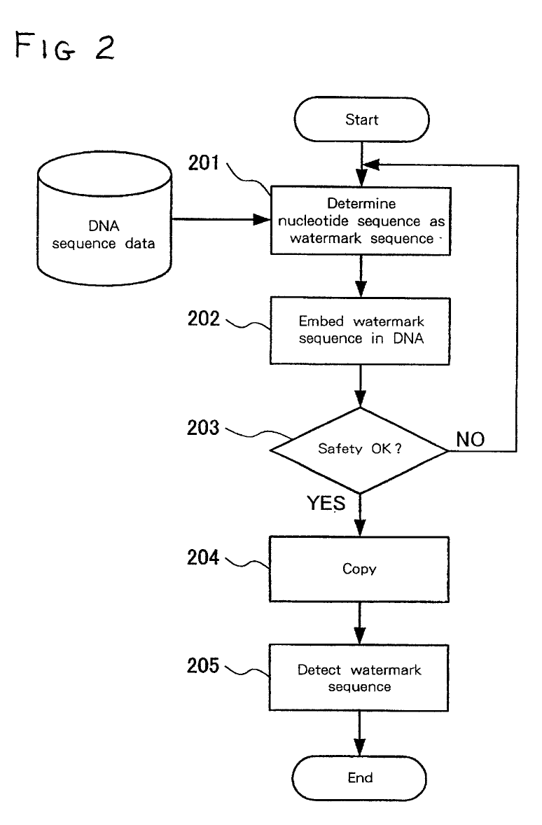

FIG. 2 is a flowchart for explaining the general processing used for the determination of a watermark sequence, its embedding in a DNA and its detection therein.

In FIG. 2, a watermark sequence is determined based on DNA sequence data (step 201). The watermark sequence determination method will be described later. Then, the watermark sequence is embedded in the DNA of an object organism (step 202). Following this, the safety of the DNA in which the watermark sequence is embedded is examined (step 203). When the safety of the DNA is confirmed, the organism including the DNA is produced, and the DNA is copied (step 204). Thereafter, the watermark sequence is detected as needed in the DNA of an organism of the same species, and the source of the DNA information carried by the organism is identified (step 205).

A technique that is similar to this invention, for preventing the illegal copying of a value-added gene, is disclosed in U.S. Pat. No. 5,723,765. This technique prevents germination of the seed at the second generation by gene manipulation. Since the seeds gathered from crops that are manipulated using this technique are not germinated, and producers must buy seeds every year, the profits of seed/seedling developing companies can be protected.

According to this technique, when seeds or seedlings of crops grown through the normal growth process of germination, blooming and pollination mature beyond the dormant stage and reach the growth point at which a second generation or leaf buds are to be developed in the seeds, protein containing a toxin, which is generated by a toxic gene, is recombined in a gene and kills the seeds.

However, with this technique, the dispersion of a toxic gene that kills embryo buds, the affect on the body of a human who ingests the toxic protein, especially as they are related to allergic reactions, or the affect on birds and insects that eat these seeds and on microorganisms, such as molds and viruses, are unknown. In the present invention, as well as this technique, a nucleotide sequence having an unknown function is embedded in the DNA; however, at the least, no nucleotide sequences that it is known apparently generate a toxic material are not embedded. IN addition, in order to control the time for the production of a toxic protein, the above technique employs a promotor that is activated when an embryo is developed. But since for the present invention, a function that depends on an organism is not required, the present invention can be easily employed for a variety of organisms.

As is described above, in this invention that is taking into account the copy toleration and the safety of the genetic information, the method used to insert a watermark sequence in a portion of a DNA other than the gene, the method for inserting a watermark sequence into the intron of the gene, and the method for employing the codon redundancy to embed watermark information have been proposed as methods for embedding a watermark sequence in the DNA. Since these watermark sequences differ in form (the insertion location, the sequence size, etc.), conditions required for an insertable watermark sequence vary, and copy toleration and ease of carrying out the method also differ. The preferred embodiments for the respective methods will now be described while referring to the accompanying drawings.

First Embodiment

First, the embodiment employing the method for the insertion of a watermark sequence in the portion of a DNA other than the gene will be described. In this embodiment, so long as a watermark sequence is detected in DNA, the source of the genetic information for the DNA can be specified. Therefore, the watermark sequence can be inserted at any location at random, and even if it is inserted in the gene portion of the DNA, the function of the watermark sequence is not lost. However, since a watermark sequence that is unrelated to the genetic information for an organism is inserted into a gene portion, the organism may be affected in some way (excludes other embodiments that will be described later, a method for inserting a watermark sequence in intron, and a method for employing the codon redundancy to embed watermark information). Therefore, the watermark sequence must be inserted into a portion of the DNA other than the gene portion. FIG. 3 is a diagram for explaining the concept of the watermark sequence insertion method according to this embodiment. For this embodiment, an explanation will now be given for the individual steps shown in FIG. 2, i.e., (1) determination of a watermark sequence, (2) embedding a watermark sequence in DNA, (3) confirmation of safety, (4) detection of a watermark sequence, and (5) toleration of a watermark sequence.

(1) Determination of a Watermark Sequence

A nucleotide sequence usable as a watermark sequence is determined. As is described above, this nucleotide sequence is a sequence (hereinafter referred to as, for example, a sequence that normally does not appear) having a pattern that normally does not appear in the DNA of a target organism for the embedding of the watermark sequence. Specifically, the nucleotide sequence is determined as follows.

Assume that the total number of bases in the DNA of an object organism is defined as N and a watermark sequence having bases n is embedded in the DNA. Since there are four types of bases (A, T, G and C), only a proposed watermark sequence WM having bases n that satisfy a condition must be selected from a set S of 4ⁿ choices. That is,

WM choices ε S(n) element count |S(n)|=4ⁿ

When V(n) denotes a sequence that is especially significant for a sequence that has a high probability of appearing in normal DNA, only the watermark sequence WM must be selected from S(n)−V(n). That is, watermark sequence WM actually employed ε S(n)−V(n). Since the number of elements in S(n), together with n, is increased in accordance with the exponential function, a sequence that does not normally appear can be found, even through it has a short length.

Assume that a watermark sequence having a length n is to be embedded in the DNA of a human (hereinafter referred to as human DNA). Since the length of the human DNA has about 30 billion bases, about 30 billion partial sequences having the length n are arranged in the human DNA. Supposing that these partial sequences are arranged evenly, there are 4ⁿ different expressions for the watermark sequences of the length n. Thus, for a partial sequence of about 20 bases, a number of base types greatly exceeding 30 billions can be obtained. Therefore, when even a nucleotide sequence of roughly 30 bases is employed, satisfactory sequences can be obtained that do not normally appear. Among these sequences, a restrictive enzyme identification sequence, or a sequence such as a promoter that does not include biological meaning can be selected as a proposed watermark sequence.

In actuality, arbitrary partial sequences in the DNA are biased. However, at present a sequence determination for the DNA of several species has been completed and it is forecast that the nucleotide sequence of the DNA will be gradually explicated for all organisms, including human beings. Based on all the nucleotide sequences, the distribution of the partial sequences can actually be understood, even though only approximately.

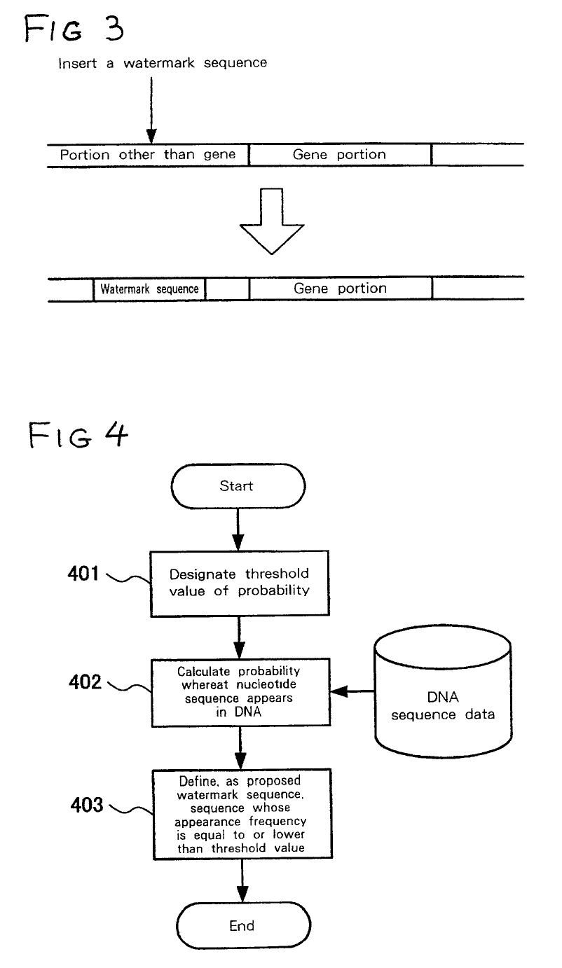

FIG. 4 is a flowchart showing the processing for calculating the appearance probability based on DNA sequence data and for determining a proposed watermark sequence. In FIG. 4, the threshold value of a probability is set to guarantee that a nucleotide sequence selected as a watermark sequence is a sequence that does not normally appear in the DNA of an object organism (step 401). Then, the DNA sequence data are employed to calculate the probability whereat a predetermined nucleotide sequence will appear in the DNA (step 402). When the probability is smaller than the threshold value at step 401, the pertinent sequence is defined as a proposed watermark sequence (step 403). If the overall nucleotide sequences in the DNA are well known, the probability that the sequence does not normally appear can be calculated approximately, based on these sequences.

For the probability calculation method to guarantee that a predetermined nucleotide sequence does not normally appear in the DNA, the process for determining a watermark sequence having a length of six bases will be described by using simple pseudo data.

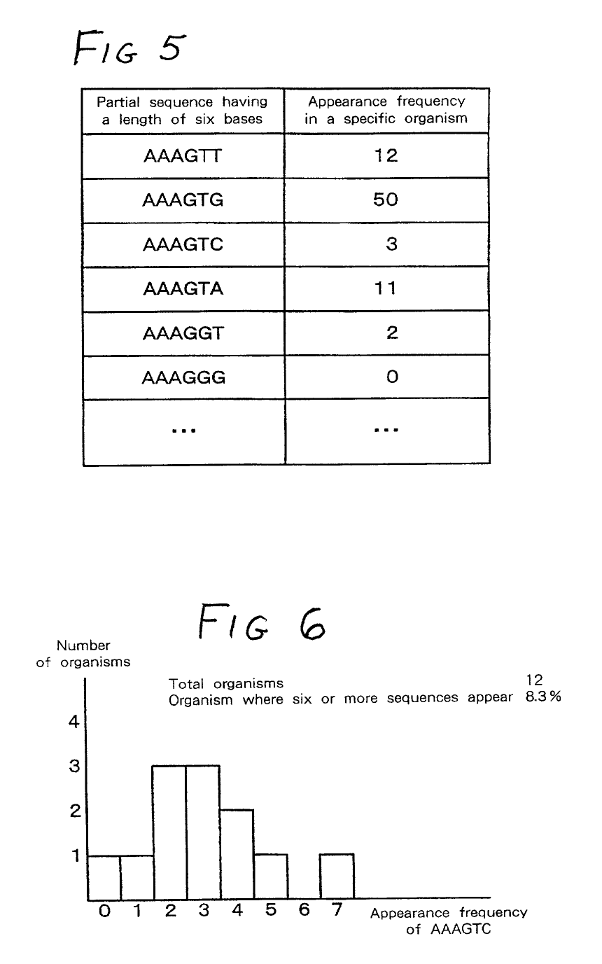

First, to determine the proposed watermark sequences, in one organism, a frequency distribution of nucleotide sequences having a length of six bases is employed. FIG. 5 is a diagram showing an example frequency distribution for the nucleotide sequences. Assume that AAAGTC is selected as a proposed watermark sequence. Since the frequency of AAAGTC is three, if AAAGTC sequences of more than three are embedded to the DNA, the nucleotide sequence AAAGTC can be employed as a watermark sequence. However, when the organism in which the watermark sequence being embedded is mated with an organism with no watermark sequence, the number of watermark sequences in an organism obtained by one mating is reduced about half because of meiosis. To avoid this phenomenon, multiple watermark sequences must be embedded in the DNA. Further, destruction of a watermark sequence due to gene mutation, or coincident generation of the same nucleotide sequence as the watermark sequence must be taken into account. Therefore, the number of watermark sequences should be determined while taking into account the fact that the frequency of the appearance of the nucleotide sequences differs in organisms due to gene mutation, etc.

As the method for taking into account a difference in the frequencies of the appearance of the nucleotide sequences among organisms, DNA sequence data are collected for as many organisms as possible, and the number of organisms for each frequency of the appearance of the nucleotide sequences can be employed as a frequency distribution table. FIG. 6 is a graph showing a frequency distribution table for the frequency of the appearance of the number of organisms relative to the nucleotide sequence AAAGTC.

In samples taken from 12 organisms in FIG. 6, one type of organism contains six or more AAAGTC sequences, 8.3% of the total. Therefore, when AAAGTC is employed as a watermark sequence and when six or more sequences are embedded in the DNA of one organism, from the distribution of pseudo data in FIG. 6, the watermark sequence will be detected in 8.3% of organisms wherein the watermark sequences were not embedded.

In this case, it can be understood that the nucleotide sequence AAAGTC functions as a watermark sequence with an error rate of 8.3%.

Further, when one kind or multiple kinds of sequences are embedded in multiple locations, the probability whereat the same nucleotide sequence as the watermark sequence will occur due to gene mutation, and the probability whereat the watermark sequence will be destroyed can be reduced. For example, when many of one kind of watermark sequences are embedded, the probability that all the watermark sequences, equivalent to the number of detected organisms, will be changed is very low.

The thus obtained probability is employed to determine a watermark sequence that can satisfy a requested probability. An explanation will be given, using pseudo data and with a protection period of 10 years, for a case wherein the source of a value-added gene, which was intentionally generated, is specified by using the watermark sequence in order to prevent the illegal employment of the value-added gene. Assume that an organism is the same species as an organism in which the watermark sequence is to be embedded, and that an estimated 1000 organisms will be present during the protection period of 10 years.

As is apparent from the frequency distribution in FIG. 6, 8.3% is the probability (error rate) when six nucleotide sequences AAAGTC are embedded in the DNA and when six or more of the nucleotide sequences are detected in an organism wherein the pertinent sequence is not intentionally embedded. Similarly, 0.02% is the error rate when ten nucleotide sequences AAAGGT are embedded, and 0.001% is the error rate when eight nucleotide sequences AAAGGG are embedded.

As watermark sequences, six nucleotide sequences AAAGTC, ten nucleotide sequences AAAGGT and eight nucleotide sequences AAAGGG are embedded in the DNA of a specific organism, and the probability is calculated as an independent phenomenon. In this case, for an organism other than that wherein these watermark sequences are intentionally embedded, the probability that all of these nucleotide sequences will be found at a frequency higher than a given frequency is

8.3×0.02×0.001=0.000166(%).

Therefore, it can be said that, of the total of 1000 organisms that will be present during the protection period of 10 years, an organism that coincidentally has the same sequence will rarely be encountered.

When a great number of watermark sequences are embedded, a target DNA can be divided into segments by a restriction enzyme and these segments can be detected by using a DNA chip, so that the number of embedded watermark sequences can roughly be obtained. Therefore, a method can be employed whereby, if statistically the number of embedded watermark sequences is significantly large, it can be ascertained that the watermark sequence has been inserted. When multiple kinds of nucleotide sequences are to be inserted into a DNA as watermark sequences, the amount of information to be added to the DNA can be increased by managing the combination of watermark sequences.

(2) Embedding a Watermark Sequence in DNA

Using a vector, the watermark sequence can be comparatively easily embedded in the DNA.

However, according to this method, the watermark sequence is inserted at random at locations in the DNA. Since the embedding location can not be designated, the watermark sequence may be inserted into a gene portion rather than into a targeted portion other than a gene. Thus, the confirmation of safety, which will be described later, is indispensable.

(3) Confirmation of Safety

As is described above, when a vector is employed to embed a watermark sequence, the embedding location can not be designated, and the watermark sequence may be inserted into a gene portion. Therefore, the safety of an organism wherein the watermark sequence has been embedded in DNA must be confirmed. For this process, whether the watermark sequence has been inserted into a portion of the DNA other than the gene portion is not determined; however, in this embodiment, so long as the watermark sequence is detected in the DNA, the function of the watermark sequence can be demonstrated, regardless of its embedded location. As a result, the safety of the organism can be satisfactorily. The standard for safety should be determined in accordance with the function of the value-added gene that is to be protected (the illegal copying of which should be prevented). This requires a social agreement, but if the value-added gene to be protected is socially approved, it is assumed that a watermark sequence providing the same safety can also be approved.

For the confirmation of safety, an organism should be used for the testing that is conducted.

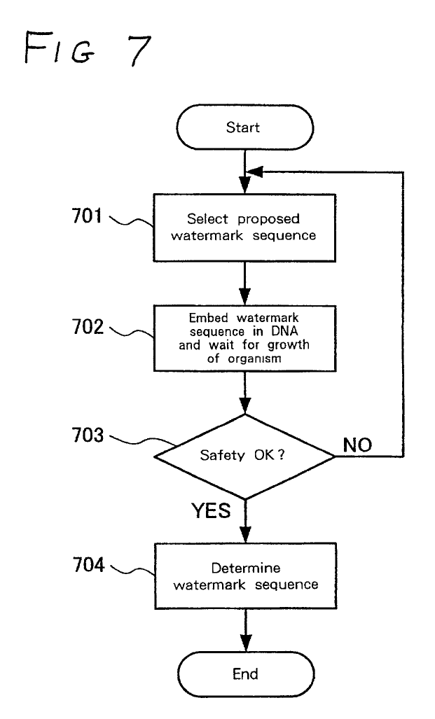

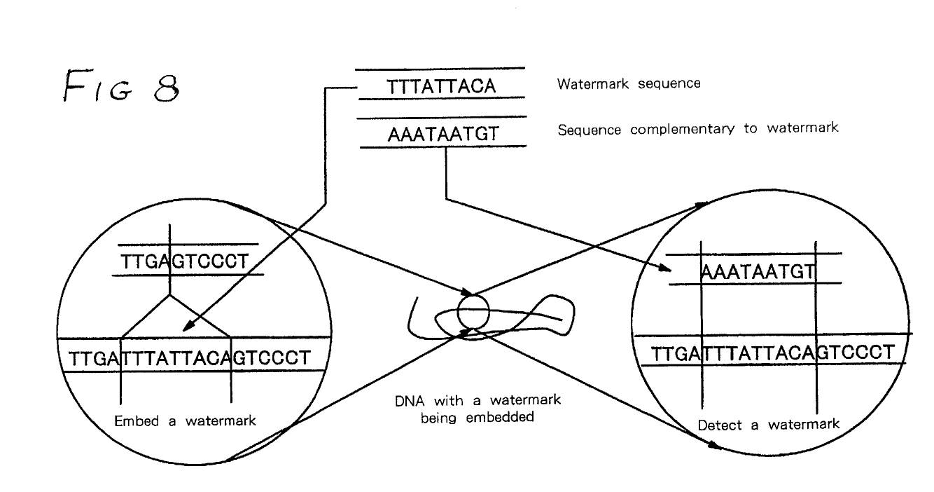

FIG. 7 is a flowchart showing the processing performed to confirm the safety of a watermark sequence. In FIG. 7, of a number of proposed watermark sequences, a single arbitrary watermark sequence is selected (step 701). The selected watermark sequence is embedded in the DNA of a predetermined organism and the procedure is paused while the organism is growing (step 702). Then, the safety of the organism that has grown is examined, and if the result is not satisfactory, another watermark sequence choice is selected and process is repeated (step 703). If the safety is confirmed, however, the pertinent sequence choice is determined to be a watermark sequence (step 704).

(4) Detection of Watermark Sequence

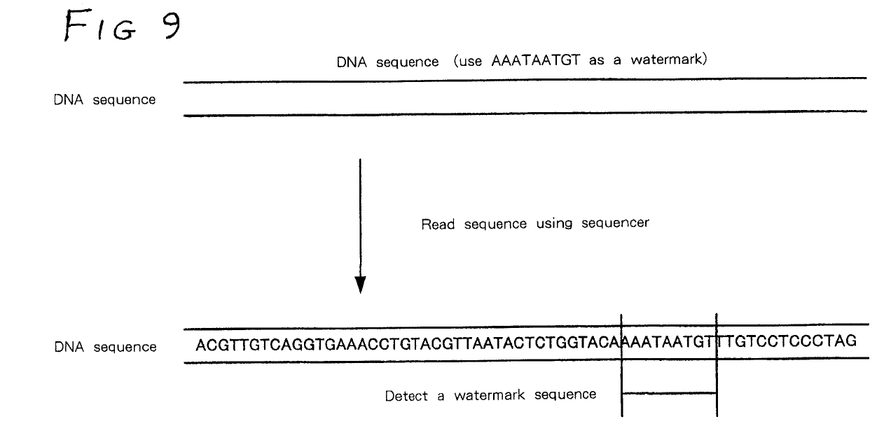

A nucleotide sequence that is complementary to an embedded watermark sequence can be employed to detect a watermark sequence in DNA. FIG. 8 is a diagram showing the state wherein a watermark sequence is detected using the complementary nucleotide sequence.

In FIG. 8, the watermark sequence TTTATTACA is embedded in DNA, and the nucleotide sequence AAATAATGT, which for this watermark sequence is complementary, is employed to detect the watermark sequence. Further, when the DNA to be searched for is extracted, and the nucleotide sequence of the DNA is read using the sequencer, if the watermark sequence is embedded in the DNA it can be detected. FIG. 9 is a diagram showing the state wherein the nucleotide sequence of the DNA is read by using the sequencer, and the watermark sequence AAATAATGT is detected.

(5) Toleration of a Watermark Sequence

In order that watermark information evidence copy toleration, the watermark sequence must be copied, and thus not be deteriorated, when the DNA is copied. In this embodiment, relative to the copying of all of the DNA due to breeding, copying due to cell transplantation, copying due to extraction of a chromosome, or copying specifically performed by removing a region in the DNA, to include the watermark sequence, a watermark sequence that is inserted at random in DNA locations other than a gene can be copied when the DNA (or a nucleotide sequence, one part of the DNA) is copied, without being degraded. In other words, the toleration is maintained by the watermark information. However, when the portion extracted of the DNA that has been copied is so small that a watermark sequence is probably not included, the watermark sequence is not copied to the nucleotide sequence copy. Therefore, for such copying, the watermark information in this embodiment does not exhibit toleration.

When the protein code region is transcribed into mRNA in the process for synthesizing genes to obtain protein, a portion other than the gene portion is not present, so that the watermark sequence is not included. Thus, when the gene is copied in the mRNA state, in this embodiment the watermark information does not exhibit tolerance.

Second Embodiment

An explanation will now be given for an embodiment that uses a method for the insertion of a watermark sequence into the intron of a gene to be protected.

As is described above, for a higher organism the mRNA obtained by transcribing the protein code region from the DNA consists of exons that are to be finally translated into amino acid and intron that is to be removed during the process (primary mRNA). Therefore, while taking into account the affect of an organism into which a watermark sequence has been inserted, the watermark sequence can be embedded in the intron that is not employed for the synthesis of protein.

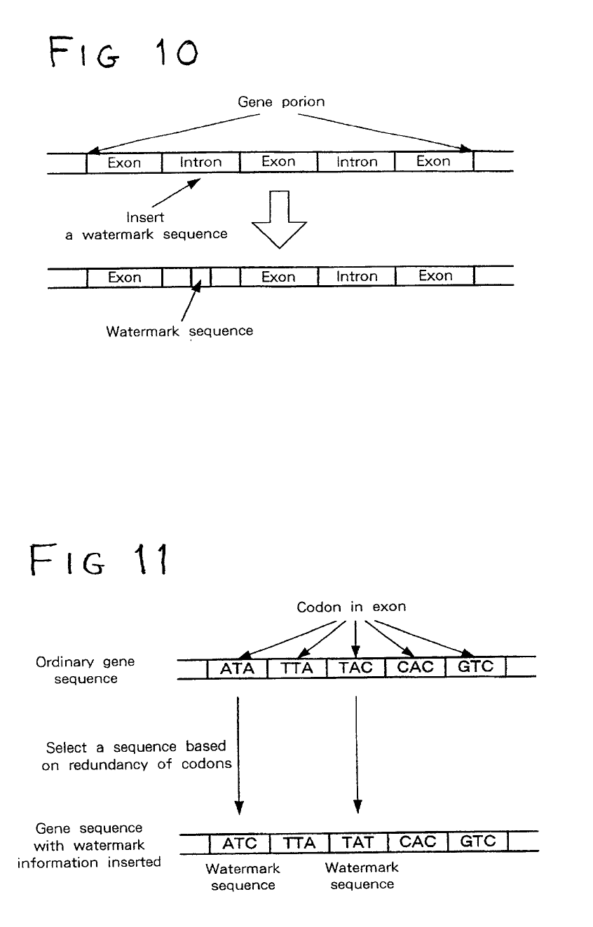

According to the embodiment, since the watermark sequence is embedded in the gene portion of the DNA, an advantage is that for protection the watermark sequence can itself be embedded into a value-added gene. FIG. 10 is a diagram for explaining the concept of the method used for the insertion of a watermark sequence according to this embodiment. For this embodiment, an explanation will now be given for the steps in FIG. 2, i.e., (1) the determination of a watermark sequence, (2) the embedding of a watermark sequence in DNA, (3) the confirmation of safety and (4) the detection of a watermark sequence, and (5) the toleration of a watermark sequence.

(1) Determination of a Watermark Sequence

A nucleotide sequence that can be employed as a watermark sequence is determined. This nucleotide sequence is one that does not normally appear in the intron of the gene portion in the DNA of a targeted organism in which the watermark sequence is to be embedded. The same method as is used in the first embodiment is employed to determine the watermark sequence. However, while in the first embodiment a sequence that overall does not normally appear in the nucleotide sequences of the DNA is employed, in this embodiment, a sequence that does not normally appear in the intron of the gene is employed.

As described in the first embodiment, for a nucleotide sequence to be used as a watermark sequence, it must not be biologically significant.

If the genetic sequence of the DNA of the target organism is already known, this genetic sequence can be employed to calculate the approximate probability that the sequence will not normally appear in the intron. For calculation of this probability, the frequency distribution used in the first embodiment can be employed for the sequences in the introns that are collected from many organisms.

Further, when one or several kinds of watermark sequences are embedded in multiple introns, the probability whereat the same nucleotide sequence as the watermark sequence will occur due to gene mutation and the probability that the watermark sequence will be destroyed can be reduced. When multiple kinds of nucleotide sequences are inserted, management for the combinations of these watermark sequences is provided, so that the amount of information that is added to the DNA can be increased.

(2) Embedding a Watermark Sequence in DNA

In the process for synthesizing genes (including the exon and the intron), when a nucleotide sequence that is determined to be a watermark sequence is inserted into a desired location in an intron, the watermark sequence can be embedded in the DNA. Preferably, the genes to be synthesized should be value-added genes that must be protected; however, they may be other genes. Furthermore, if a vector can be employed to specify the location of the intron in the gene and to embed the nucleotide sequence therein, the watermark sequence can be embedded in the intron of a desired gene.

In order to embed the watermark sequence using the method of this embodiment, it is necessary to find the intron portion in the gene. The splicing for the removal of the intron from a gene is effected by a spliceosome. The reason for this is that it is known that the nucleotide sequence included in the spliceosome is easily coupled with the nucleotide sequence of an intron start portion. Thus, as one method, the nucleotide sequence included in the spliceosome can be employed to designate the intron portion of a gene.

(3) Confirmation of Safety

As is described above, the intron is a portion removed by splicing when the gene is translated into amino acid. However, since a watermark sequence that is not related to the genetic information for an organism is inserted into the gene portion, according to this embodiment it is also necessary for the safety of the organism in which the watermark sequence is embedded to be confirmed.

As in the first embodiment, the safety standard should be determined in accordance with the function of the value-added gene to be protected.

Furthermore, for confirmation of the safety, an experiment using an organism must be conducted. The procedures for confirming the safety of the watermark sequence are performed in the same manner as in the first embodiment in FIG. 7.

(4) Detection of Watermark Sequence

As in the first embodiment, a method for employing a nucleotide sequence that for the embedded watermark sequence is complementary, or a method for employing a sequencer to read the nucleotide sequence in the DNA can be employed to detect the watermark sequence.

(5) Toleration of a Watermark Sequence

As in the first embodiment, the toleration evidenced by the watermark information in this embodiment is relative to the copying of the entire DNA, the copying using cell transplantation, the copying by the extraction of a chromosome, or the copying especially performed by removing a region in the DNA that includes the watermark sequence. Even when the portion extracted from the DNA is copied, so long as the gene is included in the portion, the intron portion will always be copied. Therefore, so long as the copying is performed as gene units, the toleration evidenced by the watermark information in this embodiment is adequate. The same thing applies in a case wherein the DNA is copied in a state wherein the protein code region is transcribed into the mRNA during the synthesization of genes to obtain protein.

However, for a higher organism, the intron portion is removed by splicing before the primary mRNA is translated into amino acid, and the watermark information in this embodiment does not possess the relative toleration for the copying from the mRNA after the splicing.

Third Embodiment

An explanation will now be given for an embodiment using the method for which codon redundancy is employed to embed watermark information.

As is described above, the nucleotide sequence of DNA is coded in amino acid using codon units composed of three characters. However, since 64 (=4³) different three base combinations can be formed for 20 kinds of amino acids in an organism, and multiple codon codes may be present for one type of amino acid, so that the watermark information can be embedded in this redundant portion.

The correlation between the codons to be translated into amino acid during the protein synthesis process and the amino acid is provided by the codon table. By referring to the codon table in FIG. 13, it is apparent that multiple codons are correlated with one amino acid, and that the redundancy is mainly located at the third character of the codon. Thus, within the range permitted according to the codon table, i.e., so long as the codons are correlated with the same amino acid, each codon in the exon of a gene to be protected can be freely replaced by another base. This degree of freedom is employed to embed the watermark information. Therefore, in this embodiment, the sequence of codons selected for the insertion of the watermark information serves as a watermark sequence.

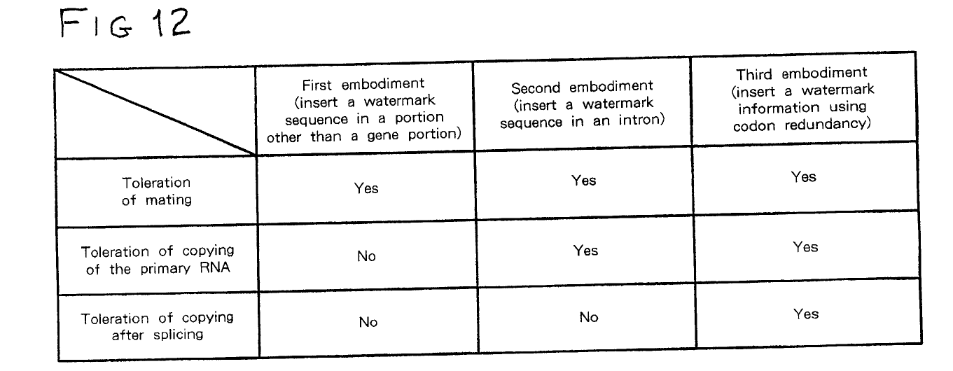

According to the present invention, an advantage is that the watermark information can be embedded in the exons in the gene portions of the DNA. FIG. 11 is a diagram for explaining the concept of the watermark sequence insertion method according to the embodiment.

For this embodiment, an explanation will now be given for the steps in FIG. 2, i.e., (1) the determination of a watermark sequence, (2) the embedding a watermark sequence in a DNA, (3) the confirmation of safety and (4) the detection of a watermark sequence, and (5) the toleration of a watermark sequence.

(1) Determination of a Watermark Sequence

As is described above, there are multiple codes (codons) of nucleotide sequences that correspond to one amino acid. Thus, when the codons corresponding to a predetermined amino acid are intentionally selected, additional information can be embedded directly in the gene, without changing the meaning of the sequence, which is the code of useful protein (an amino acid sequence that has been coded). In this process, at the present time, the strict replacement, such as the replacement of only a desired codon (the base in one part of the codon) in the DNA, seems to be technically difficult.

However, instead of directly rewriting the nucleotide sequence in the DNA, when new protein is designed at the level of amino acid, or when the amino acid sequence that is coded using an exotic gene for the insertion is read and a corresponding DNA is designed, the watermark information can be embedded in the process for the replacement of the amino acid in the codon.

It should be noted that the employment of the codons differs in accordance with the species of organisms, and is normally biased. Thus, when codons that are less frequently employed are used for a specific organism, there are few corresponding tRNAs, so that the transcription efficiency will be reduced and the expected function of protein will be lowered.

Therefore, a method is employed that uses the two codons whose appearance frequencies are the highest and the second highest, and to write information that correlates these codons with binary data (0, 1). N codons are employed, and whether each codon (since there is only one kind of codon corresponding to methionine, this is excluded) corresponds to 0 or 1 is determined. Then, when the information is read, the binary data string (hereinafter referred to as a bit string) having a length N is obtained. Thereafter, the watermark information is written using this bit string.

This method will now be described more in detail. In order to affect the efficiency of the synthesis of protein as little as possible, the two codons whose appearance frequencies are the highest and the second highest are selected for the amino acids other than methionine, and are allocated values of “0” or “1.” All the portions that can be used for coding can be employed for the exons in genes in order to embed the information. Further, codons to be used and codons not to be used may be distinguished by using a pseudo random key, and the same key may be employed for the detection of the extraction of information only from codons that are used for embedding.

There are two problems with this embodiment. As one, a false positive error may occur whereby, in accordance with the above described rule, some message will also be extracted from the gene of an organism in which no information has been embedded. That is, when a bit string “1001” is extracted, there is no means for ascertaining whether the bit string was intentionally embedded information or a combination that occurred naturally.

As another problem, a false negative error may occur whereby the rule of repetition may be destroyed because some mutation has occurred in the genes in which information has been embedded, and a bit string that represents the watermark information can not be detected.

As one method for resolving the problem that is due to a false positive error, a method for respectively embedding a message can be employed. If the probability whereat the repetition of the message is sufficiently low for a gene in which no information has been embedded, it can be ascertained that information has been embedded in a DNA in which the repetition of the bit string is detected. The probability of the occurrence of a false positive error is obtained as follows.

For simplifying the explanation, only one type of amino acid, A, is employed for coding. From among codons that synthesize the amino acid A, assume that the codon whose appearance frequency in an organism is the highest is defined as CO (employment probability PC), and the codon whose appearance frequency is the second highest is defined as C1 (employment probability P1). Further, assume that the bit “0” is allocated to C0 and the bit “1” is allocated to C1, and that the total number N of C0 and C1 are included in the exon of a target gene for information embedding. In this example, the watermark information that is represented by a predetermined bit string consisting of C0 and C1 is respectively embedded m times. In this case, information consisting of n bits (N=mn) can be embedded.

Under the above assumption, the probability that a false positive error will occur is represented by equation 1.

$$( false\_positive \_error ) = {\sum }_{k = 0}^{n} ( \begin{matrix}n \\ k\end{matrix} ) {( P 0 )}^{k} {( P 1 )}^{n - k} \\ wherein ( \begin{matrix}n \\ k\end{matrix} ) = \frac{n !}{k ! ( n - k ) !} \qquad \text{[Equation 1]}$$

n multiple kinds of amino acids are employed for coding, the frequency whereat each kind of amino acid appears in an exon can be substituted into equation 1 to obtain the probability.

Furthermore, when s bits (s<n) of the n bits are employed for the message and the remaining (n−s) bits are employed as an error correction sign, the probability of the occurrence of a false negative error can also be reduced considerably.

In the above explanation, from among the codons that synthesize the amino acid A, the two codons C0 and C1, whose appearance frequencies are the highest and the second highest, are assigned bits 0 and 1. However, when codons other than C0 and C1 are replaced with 0 and 1, the amount of information to be embedded can be increased.

(2) Embedding a Watermark Sequence in DNA

When, during the process for the synthesization of genes, bases constituting a specific codon are appropriately selected and a bit string representing watermark information is prepared, the watermark information can be embedded in the DNA. Further, when the replacement of each base or the replacement of the bases for each codon is performed as an extension of the gene synthesis technique, the watermark information can also be embedded in the DNA.

(3) Confirmation of Safety

A gene in which the watermark information is embedded using the method of the embodiment synthesizes the same protein as the gene a target organism originally included. However, since each codon in a gene is artificially rewritten within the range of the redundancy, it is difficult to say there is no side effect affecting an organism. Therefore, also in this embodiment, the confirmation of safety is required for an organism in which the watermark information is embedded. As in the first embodiment, the safety standard should be determined in accordance with the function of the value-added gene to be protected.

For the confirmation of safety, an experiment using an organism should be conducted. The procedures for confirming the safety of a watermark sequence are performed in the same manner as in the first embodiment in FIG. 7.

(4) Detection of a Watermark Sequence

As in the first embodiment, the method for employing a nucleotide sequence complements the embedded watermark sequence, or a method for employing a sequencer to read the nucleotide sequence in the DNA can be employed to detect the watermark sequence.

(5) Toleration of a Watermark Sequence

As in the first and the second embodiments, the watermark information in this embodiment has a toleration relative to the copying of the overall DNA, the copying using cell transplantation, the copying using the extraction of a chromosome, or the copying that is especially performed by removing a region in the DNA that includes the watermark sequence.

In addition, as in the second embodiment, even when the portion extracted from the DNA is copied, so long as the gene is included in the portion, the intron portion is always copied. Therefore, so long as the copying is performed as units of genes, in this embodiment the toleration of watermark information is ensured. The same thing is applicable for a case wherein the DNA is copied in a state wherein the protein code region is transcribed into the mRNA during the synthesization of genes to obtain protein.

Furthermore, in this embodiment, since watermark information is embedded in the exons of genes, the watermark information is also included in the mRNA that is finally translated into the amino acid. Thus, the watermark information in this embodiment possesses a toleration that is also relative to the copying of the mRNA after the splicing has been performed.

The watermark information that is embedded, in each of the embodiments, in the DNA in the above described manner can be employed as information to determine the source of genetic information, in accordance with the toleration attributable to the information.

FIG. 12 is a table showing the toleration of the watermark sequence for the first, the second and the third embodiments relative to the individual copying methods.

In FIG. 12, all the watermark sequences for the first, the second and the third embodiments have toleration attributable to the mating. The watermark sequences in the second and the third embodiments have toleration relative to the copying of the primary RNA. And the watermark sequence in the third embodiment has toleration relative to the copying from the mRNA after the splicing.

When the watermark sequence is detected and analyzed, it can be confirmed that a value-added gene that is included with the watermark sequence in a gene is a copy of a specific gene. Further, when the right to produce or to copy this value-added gene is restricted by the establishment of a contract, or by another means, it can be determined whether the copy of the gene is legal, and illegal copying can be prevented.

As is described above, according to the present invention, since predetermined information is embedded in the nucleotide sequence of DNA, the source of the genetic information in DNA can be identified.

Further, according to the present invention, since information that is intentionally embedded in the sequence of nucleotides making up DNA is detected and analyzed, it is possible to determine whether a predetermined gene owned by a predetermined organism is a copy of a specific gene.

In addition, according to the present invention, since a check is performed to determine whether a predetermined gene owned by a predetermined organism is a copy of a specific gene, the illegal copying of the specific gene by a third party can be prevented.

What is claimed is:

1. A method for writing information in DNA comprising the steps of:
correlating the pattern of a nucleotide sequence, which normally does not appear in a portion of said DNA other than a gene, with identification information for identifying a source of predetermined genetic information belonging to said DNA; and
embedding, in said portion of said DNA other than said gene, said nucleotide sequence that is correlated with said identification information.

2. A method for writing information in DNA comprising the steps of:
correlating the pattern of a nucleotide sequence, which normally does not appear in the intron of a DNA, with identification information for identifying the source of predetermined genetic information owned by said DNA; and
embedding, in said intron of said DNA, said nucleotide sequence that is correlated with said identification information.

3. A method for writing information in DNA comprising the steps of:
employing redundancy for a codon to be translated into amino acid so that multiple codons to be translated into the same amino acid are correlated with binary data; and
arranging, in the exon of a gene, said codons that are correlated with said binary data, and to thus form a data sequence representing predetermined information.

4. A method for identifying the source of genetic information in DNA comprising the steps of:
obtaining DNA from an arbitrary organism of the same species as an organism wherein a source identification nucleotide sequence, for designating the source of genetic information, is embedded into said DNA; and
employing as said source identification nucleotide sequence a complementary nucleotide sequence in order to determine whether said source identification nucleotide sequence is present in said DNA of said arbitrary organism.

5. A DNA to which information is added comprising:
a gene portion including genetic information; and
a portion, other than said gene portion, including no genetic information,
wherein said portion other than said gene portion includes a nucleotide sequence that is correlated with source identification information and specifies a source of genetic information that is transmitted by said gene portion.

6. DNA wherein a gene portion that includes genetic information also includes exon that is translated into amino acid when protein is to be synthesized, and intron that is removed when protein is to be synthesized; and wherein said intron includes a nucleotide sequence that is correlated with source identification information for designating a source of genetic information that is included in said exon.

7. DNA comprising:
multiple kinds of codons that are correlated with said binary data using said codon redundancy and are translated into amino acid,
wherein binary data are used to correlate said codon array in said gene portion with a data sequence that represents predetermined information.

8. DNA wherein a special sequence that is intentionally designed is included as a part of a nucleotide sequence; wherein said special sequence is correlated with source identification information for designating the source of genetic information included in said DNA; and wherein said special sequence is embedded in said DNA so as not to affect the transmission of said genetic information included in said DNA.

9. DNA according to claim 8, wherein multiple of said special sequences are embedded at predetermined locations of said DNA.

10. DNA according to claim 8, wherein said special sequences having multiple types of patterns are embedded at predetermined locations of said DNA.

11. A nucleotide sequence constituting one part of DNA, being correlated with source identification information for designating a source of genetic information in DNA, and being embedded in said DNA so as not to affect transmission of said genetic information in said DNA.

12. A cell constituting an organism, wherein DNA included in said cell comprises:
a gene portion including genetic information, and
a portion, other than said gene portion, including no genetic information; and wherein said portion other than said gene portion includes a nucleotide sequence that is correlated with source identification information and specifies a source of genetic information that is transmitted by said gene portion.

13. A cell constituting an organism, wherein a gene portion of DNA included in said cell includes genetic information also includes exon that is translated into amino acid when protein is to be synthesized, and intron that is removed when protein is to be synthesized; and wherein said intron includes a nucleotide sequence that is correlated with source identification information for designating a source of genetic information that is included in said exon.

14. A cell constituting an organism, wherein DNA contained in said cell includes multiple kinds of codons that are correlated with said binary data using said codon redundancy and are translated into amino acid; and wherein binary data are used to correlate said codon array in said gene portion with a data sequence that represents predetermined information.
